(19) United States
(12) Patent Application Publication
(10) Pub. No.: US 2024/0010607 A1
(43) Pub. Date: Jan. 11, 2024

(54) NEW SYNTHESIS OF L-PHENYLALANINE ESTER

(71) Applicant: DSM IP ASSETS B.V., Heerlen (NL)

(72) Inventors: Werner BONRATH, Kaiseraugst (CH); Alexander GAA, Kaiseraugst (CH); Ralph HAERTER, Kaiseraugst (CH)

(21) Appl. No.: 18/253,124

(22) PCT Filed: Nov. 9, 2021
(86) PCT No.: PCT/EP2021/081031

§ 371 Date: May 16, 2023

(30) Foreign Application Priority Data
Nov. 18, 2020 (EP) 20208402.6

(51) Int. Cl. C07C 227/18

(52) U.S. Cl. CPC C07C 227/18

(57) ABSTRACT

The present invention relates to a process for producing L-phenylalanine ester which comprises the reaction of L-phenylalanine with an alcohol in the presence of thionyl chloride.

The present invention relates to a process for producing L-phenylalanine ester.

L-Phenylalanine ester is an important amino acid ester, which can be used as such or which can be used as an intermediate in organic synthesis.

Because L-phenylalanine ester is an important compound there is always a need for improved ways to produce it.

Surprisingly, it was found that when an alcohol is used as a solvent, the reaction results in excellent yield and the work-up is easy. The reaction can be carried out at room temperature or slightly elevated temperatures.

L-Phenylalanine ester is the following compound of formula (I)

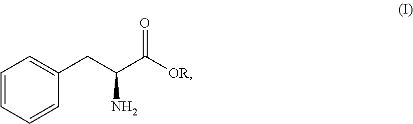

wherein R is a C₁-C₆ alkyl moiety, which can be linear or branched.

The new and improved synthesis of L-phenylalanine ester allows to obtain L-phenylalanine ester in an excellent yield.

Therefore the present invention relates to process (P) to produce L-phenylalanine ester, which is the compound of formula (I)

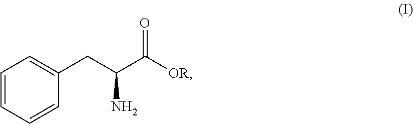

wherein R is a C₁-C₆ alkyl moiety, which can be linear or branched, characterised in that a compound of formula (II)

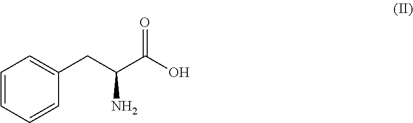 [SMILES: N[C@@H](Cc1ccccc1)C(=O)O]

is reacted with an alcohol of formula (III)

R—OH (III),

wherein R has the same meanings as defined for the compound of formula (I) in the presence of thionyl chloride.

One of the advantages of the process according to the present invention is that the alcohol of formula (III) serves as reactant as well as solvent.

As stated above the process according to the present invention is carried out in the presence of at least one alcohol of formula (III).

The alcohol (or the mixture of alcohols) are the ones of formula (III)

R—OH (III),

wherein R is a C₁-C₆ alkyl moiety, which can be linear or branched.

The alcohols of formula (III) can be primary, secondary or tertiary alcohols.

More preferred are alcohols such as methanol, ethanol, propanol, butanol, pentanol, hexanol, sec-hexanol, sec-butanol and tert-butanol.

Most preferred is ethanol.

Therefore the present invention relates to process (P1), which is process (P), wherein the alcohol of formula (III) is a primary, secondary or tertiary alcohols.

Therefore the present invention relates to process (P1′), which is process (P), wherein the at least one alcohol is chosen from the group consisting of methanol, ethanol, propanol, butanol, pentanol, hexanol, sec-hexanol, sec-butanol and tert-butanol.

Therefore the present invention relates to process (P1″), which is process (P), wherein the at alcohol is ethanol.

The process according to the present invention can be carried without any (additional) solvent. The alcohol of formula (III) can serve as solvent as well. Therefore, the alcohol of formula (III) is usually added in excess in regard of the compound of formula (II).

The at least one alcohol (compound of formula (III)) is used in an excess in view of the compound of formula (II). The molar ratio of the alcohol to the compound of formula (II) is usually at least 2:1. The upper limit is not essential for the invention. Usually it is up to 100:1. A preferred molar ratio of the alcohol to the compound of formula (II) is usually at least 10:1 to 50:1.

Therefore the present invention relates to process (P2), which is process (P), (P1), (P1′), (P1″) or (P1′″), wherein the molar ratio of the alcohol (compound of formula (III)) to the compound of formula (II) is at least 2:1.

Therefore the present invention relates to process (P2′), which is process (P), (P1), (P1′), (P1″) or (P1′″), wherein the molar ratio of the alcohol (compound of formula (III)) to the compound of formula (II) is 2:1 to 100:1.

Therefore the present invention relates to process (P2″), which is process (P), (P1), (P1′), (P1″) or (P1′″), wherein the molar ratio of the alcohol (compound of formula (III)) to the compound of formula (II) is 10:1 to 50:1.

Therefore the present invention relates to process (P3), which is process (P), (P1), (P1′), (P1″), (P1′″), (P2), (P2′) or (P2″), wherein step no further solvent (next to the at least one alcohol) is used.

It is possible to add another inert solvent to the reaction mixture of the process according to the present invention.

Therefore the present invention relates to process (P4), which is process (P), (P1), (P1′), (P1″), (P1″′), (P2), (P2′) or (P2″), wherein an inert solvent is added to the reaction mixture.

Also mixtures of more than one alcohol of formula (III) can be used in the process according to the present invention can be used. When using such a mixture, this will result in a mixture of the product of the process of the present invention.

Usually the process according to the present invention is carried out at an elevated temperature. A suitable temperature range for this step of the process according to the present invention goes from 30° C.-150° C. (preferably from 40° C.-130° C., more preferably 50° C.-120° C.).

Therefore the present invention relates to process (P5), which is process (P), (P1), (P1′), (P1″), (P1′″), (P2), (P2′), (P2″), (P3) or (P4), wherein the process according to the present invention is carried out at an elevated temperature.

Therefore the present invention relates to process (P5′), which is process (P5), wherein the temperature goes from 30° C.-150° C.

Therefore the present invention relates to process (P5″), which is process (P5), wherein the temperature goes from 40° C.-130° C.

Therefore the present invention relates to process (P5′″), which is process (P5), wherein the temperature goes from 50° C.-120° C.

Usually the process according to the present invention is carried out at ambient pressure.

Therefore the present invention relates to process (P6), which is process (P), (P1), (P1′), (P1″), (P1″′), (P2), (P2′), (P2″), (P3), (P4), (P5), (P5′), (P5″) or (P5″′), wherein the process is carried out at an ambient pressure.

In the process according to the present invention thionyl chloride is usually added to the reaction mixture in equimolar amounts in regard to the compound of formula (II). It also possible to add thionyl chloride in a slight excess in regard to the compound of formula (II).

The molar ratio of thionyl chloride to the compound of formula (II) is 1:1 to 1.5:1.

Therefore the present invention relates to process (P7), which is process (P), (P1), (P1′), (P1″), (P1′″), (P2), (P2′), (P2″), (P3), (P4), (P5), (P5′), (P5″), (P5′″) or (P6), wherein the molar ratio of thionyl chloride to the compound of formula (II) is 1:1 to 1.5:1.

Afterwards the product (compound of formula (I)) is isolated from the reaction mixture (and optionally purified) by usual means.

L-Phenylalanine ester (compound of formula (I) is obtained in excellent yields.

The following Example illustrates the invention further without limiting it. All percentages and parts, which are given, are related to the weight and the temperatures are given in ° C., when not otherwise stated.

### EXAMPLES

### Example 1

In a 750-ml four-necked flask equipped with a KPG-stirrer, thermometer, and a reflux condenser with an argon inlet was charged with 10.2 g (60.5 mmol) L-phenylalanine and 120 ml ethanol (2055 mmol). The mixture was stirred at 400 rpm and at RT (25° C.) and then dropwise was added 4.9 ml (66.6 mmol) thionyl chloride during 15 min. The reaction mixture was refluxed 78° C. (100° C. oil) for 16 h. The mixture was cooled to 0° C. and 300 ml distilled water was added slowly at 0° C. With sodium carbonate the pH was adjusted from pH 1.7 to pH 8-10. The resulting L-phenylalanine-ester was extracted 3 times with 300 ml diethyl ether. The organic phase was dried over sodium sulfate and evaporated under reduced pressure (10 mbar, 40° C.). 9.07 g L-phenylalanine-ethyl ester with a yield of 75% with a purity of 97.17% has been obtained.

1. Process to produce a compound of formula (I) 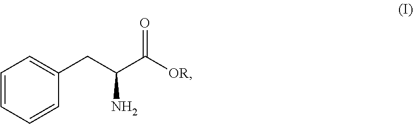
wherein R is a C1-C6 alkyl moiety, which can be linear or branched a compound of formula (II) 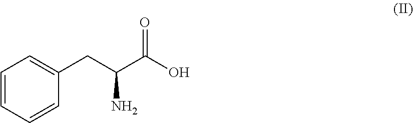 [SMILES: N[C@@H](Cc1ccccc1)C(=O)O]
is reacted with an alcohol of formula (III)
R—OH (III),
wherein R has the same meanings as defined for the compound of formula (I) in the presence of thionyl chloride.

2. Process according to claim 1, wherein the alcohol of formula (III) is a primary, secondary or tertiary alcohol.

3. Process according to claim 1, wherein the at least one alcohol is chosen from the group consisting of methanol, ethanol, propanol, butanol, pentanol, hexanol, sec-hexanol, sec-butanol and tert-butanol.

4. Process according to claim 1, wherein the alcohol is ethanol.

5. Process according to claim 1, wherein the molar ratio of the alcohol (compound of formula (III)) to the compound of formula (II) is at least 2:1.

6. Process according to claim 1, wherein the process is carried out at a temperature of from 30° C.-150° C.

7. Process according claim 1, wherein the process is carried out at an ambient pressure.

8. Process according claim 1, wherein the molar ratio of thionyl chloride to the compound of formula (II) is 1:1 to 1.5:1.
